(19) United States
(12) Patent Application Publication
(10) Pub. No.: US 2013/0196878 A1
(43) Pub. Date: Aug. 1, 2013

(54) APPARATUS AND METHOD FOR DETECTING MULTIPLEX TARGET NUCLEIC ACIDS IN REAL TIME

(71) Applicants: Bongchu Shim, Seoul (KR); Dami Kim, Seoul (KR); Seongmoon Cho, Seoul (KR); Jeankun Oh, Seoul (KR)

(72) Inventors: Bongchu Shim, Seoul (KR); Dami Kim, Seoul (KR); Seongmoon Cho, Seoul (KR); Jeankun Oh, Seoul (KR)

(73) Assignee: LG ELECTRONICS INC., Seoul (KR)

(21) Appl. No.: 13/878,040

(22) PCT Filed: Oct. 31, 2011
(86) PCT No.: PCT/KR2011/008207

§ 371 Date: Apr. 5, 2013

(30) Foreign Application Priority Data
Nov. 1, 2010 (KR) 10-2010-0107796

(51) Int. Cl. C12Q 1/68

(52) U.S. Cl. CPC C12Q 1/6834
USPC 506/9; 506/39

(57) ABSTRACT

The present invention relates to an apparatus and method for simultaneously detecting various types of target nucleic acids in real time, whereby various types of target nucleic acids can be simultaneously detected in real time to allow manufacturing an apparatus in the shape of a chip, and to efficiently detect a plurality of species of nucleic acids in a small space.

### TECHNICAL FIELD

The teachings in accordance with exemplary embodiments of this invention relate generally to an apparatus and method for detecting multiplex target nucleic acids in real time, and more particularly to an apparatus and method for simultaneously detecting various types of target nucleic acids in real time.

### BACKGROUND ART

A molecular diagnosis using nucleic acids such as DNA and RNA is a field that is a most quickly developing area in markets for in vitro diagnosis. Particularly, utilization of a molecular diagnosis of contagious diseases, capable of accomplishing a higher accuracy over that of a conventional immunoassay method due to enhanced specificity and selectivity, leading to enablement of faster diagnosis owing to a shortened period from contagion to detection, is gradually on the increase for diagnosis of viral infection such as HIV, HCV and HBV, and STDs (Sexually Transmitted Diseases) such as Chlamydia and gonorrhea.

Essence of molecular diagnosis rests on technique amplifying nucleic acids such as DNA and RNA using PCR (Polymerase Chain Reaction). The well-known PCR is a laboratory technique used to amplify and simultaneously quantify a target nucleic acid by inserting a primer designed to be selectively attached to the target nucleic acid, reaction solution containing polymerase and n NTP, and specimen of target nucleic acid as a mold through change of temperature. Methods of detecting quantity of amplification include a method using Gel electrophoresis after completion of reaction and a method using FPET (Fluorescence Resonance Energy Transfer) including TagMan (Roche), molecular beacon and scorpion for real time amplification through a probe.

Currently, the molecular diagnosis largely uses a PCR capable of detecting amplification in real time. This is because the PCR can simplify test procedures, provide a closed system capable of reducing influence caused by outside pollution and enable quantification of specimen. However, the conventional amplification and detection techniques are disadvantageously limited to amplification and detection of only one type of target nucleic acids using one chip.

As a result, a multiplex chip manufacturing technique is being developed capable of performing several types of target nucleic acids at one time. The multiplex chip manufacturing technique is also disadvantageous in that a competitive reaction generated by simultaneous reaction of several types of target nucleic acids creates errors and decreased accuracy.

### DISCLOSURE OF INVENTION

Technical Problem

The present invention is disclosed to solve the aforementioned disadvantages and/or problems and it is an object of the present invention to provide an apparatus configured to detect multiplex target nucleic acids in real time, and a method configured to simultaneously detect a plurality of types of target nucleic acids in real time using the apparatus.

Technical problems to be solved by the present invention are not restricted to the above-mentioned, and any other technical problems not mentioned so far will be clearly appreciated from the following description by skilled in the art.

Solution to Problem

An object of the invention is to solve at least one or more of the above problems and/or disadvantage in whole or in part and to provide at least the advantages described hereinafter. In order to achieve at least the above objects, in whole or in part, and in accordance with the purposes of the invention, as embodied and broadly described, and in one general aspect of the present invention, there is provided an apparatus configured to detect multiplex target nucleic acids in real time, the apparatus characterized by: a substrate; two or more target nucleic acid amplifiers including an electrode attached with a set of a forward primer for amplifying target nucleic acids and a reverse primer.

In some exemplary embodiments, the electrode is characterized by a first electrode immobilized with the forward primer, and a second electrode immobilized with the reverse primer, wherein the first and second electrodes are adjacently arranged in parallel.

In some exemplary embodiments, the two or more target nucleic acid amplifiers are characterized by an electrode attached with a set of different types of forward primers and reverse primers.

In some exemplary embodiments, the target nucleic acid amplifiers are further characterized by dNTP and DNA polymerase.

In some exemplary embodiments, the target nucleic acid amplifiers are further characterized by detectable labels

In some exemplary embodiments, the apparatus is further characterized by a voltage supplier supplying voltages of mutually different capacities to the electrode of the two or more target nucleic acid amplifiers.

In some exemplary embodiments, the apparatus is further characterized by a voltage supplier sequentially supplying a voltage to the electrode of the two or more target nucleic acid amplifiers.

In some exemplary embodiments, the apparatus is further characterized by a heat sensor arranged about the electrode immobilized with the primer set.

In some exemplary embodiments, the two or more target nucleic acid amplifiers are further characterized by a heat sensor arranged at a bottom surface of the electrode immobilized with the primer set.

In some exemplary embodiments, the apparatus is further characterized by a temperature controller controlling each temperature of the heat sensor at the two or more target nucleic acid amplifiers.

In another general aspect of the present invention, there is provided a method for detecting multiplex target nucleic acids in real time, the method characterized by: contacting various types of target nucleic acid sample to two or more target nucleic acid amplifiers of the claim 1 immobilized with a forward and reverse primer set for amplifying target nucleic acids; applying a voltage to the target nucleic acid amplifiers and controlling the applied voltage to anneal the target nucleic acids and the primer set; amplifying the target nucleic acids annealed by the primer set; and detecting amplification products of the target nucleic acids in real time.

In some exemplary embodiments, the two or more target nucleic acid amplifiers are characterized by an electrode attached with mutually different forward and reverse primer sets.

In some exemplary embodiments, the detection is characterized by an optical detection or an electrical detection.

In some exemplary embodiments, the control of voltage is characterized by individual realization at the two or more target nucleic acid amplifiers.

In some exemplary embodiments, the control of voltage is characterized by control of capacity or intensity of the voltage.

In some exemplary embodiments, the control of voltage is characterized by control of sequence of the voltage individually applied to the two or more target nucleic acid amplifiers.

In some exemplary embodiments, the two or more target nucleic acid amplifiers are further characterized by a heat sensor arranged about the electrode immobilized with the primer set.

In some exemplary embodiments, the annealing step is further characterized by individually applying heat to the heat sensor included in the two or more target nucleic acid amplifiers to perform the annealing under mutually different temperature conditions.

Advantageous Effects of Invention

The present invention has an advantageous effect in that various types of target nucleic acids can be simultaneously detected in real time to allow manufacturing the apparatus in the shape of a chip, and to efficiently detect a plurality of species of nucleic acids in a small space.

Various aspects and embodiments of the invention are described in further detail below.

The technical solution is neither intended nor should it be construed as being representative of the full extent and scope of the present invention, which these and additional aspects will become more readily apparent from the detailed description, particularly when taken together with the appended drawings. As mentioned above, the technical solution is not an extensive overview and is not intended to identify key or critical elements of the apparatuses, methods, systems, processes, and the like, or to delineate the scope of such elements. The technical solution provides a conceptual introduction in a simplified form as a prelude to the more-detailed description that follows.

### BRIEF DESCRIPTION OF DRAWINGS

The teachings of the present invention can be readily understood by considering the following detailed description in conjunction with the accompanying drawings, in which:

FIG. 1 is a plan view of an apparatus for detecting target nucleic acids in various species according to an exemplary embodiment of the present invention;

FIG. 2 is a plan view of an apparatus formed with first and second electrodes for detecting target nucleic acids in various species according to an exemplary embodiment of the present invention;

FIG. 3 is an enlarged perspective view of a target nucleic acid amplifier in an apparatus for detecting target nucleic acids in various species according to an exemplary embodiment of the present invention;

FIG. 4 is a rear view of an apparatus formed with a heat sensor for detecting target nucleic acids in various species according to an exemplary embodiment of the present invention;

FIG. 5 is a rear view of an apparatus formed with a heat sensor and first and second electrodes for detecting target nucleic acids in various species according to an exemplary embodiment of the present invention;

FIG. 6 is an enlarged perspective view of an apparatus formed with a heat sensor for detecting target nucleic acids in various species according to an exemplary embodiment of the present invention; and

FIGS. 7 and 8 are mimetic diagrams illustrating a method simultaneously detecting target nucleic acids in various species using an apparatus for detecting target nucleic acids in various species according to an exemplary embodiment of the present invention.

### BEST MODE FOR CARRYING OUT THE INVENTION

The following description is not intended to limit the invention to the form disclosed herein. Consequently, variations and modifications commensurate with the following teachings, and skill and knowledge of the relevant art are within the scope of the present invention. The embodiments described herein are further intended to explain modes known of practicing the invention and to enable others skilled in the art to utilize the invention in such, or other embodiments and with various modifications required by the particular application(s) or use(s) of the present invention.

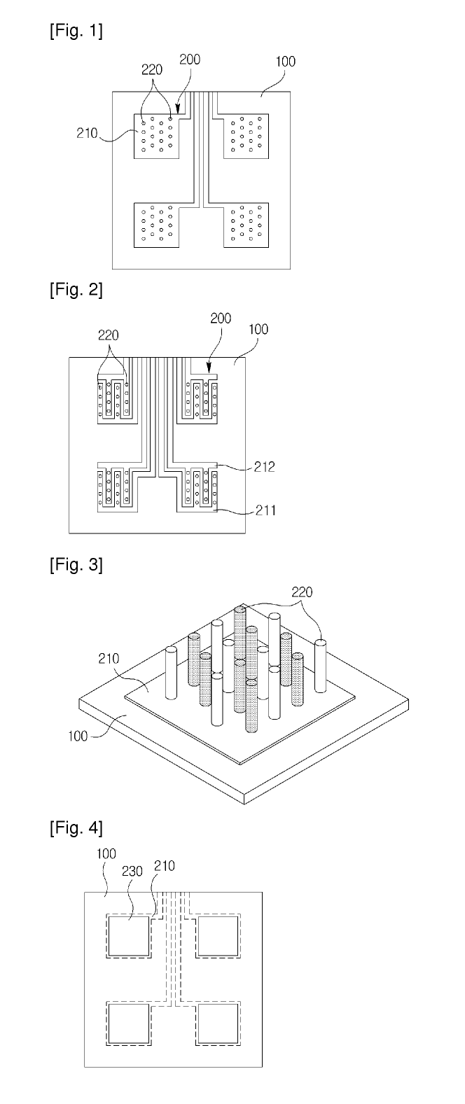

The disclosed embodiments and advantages thereof are best understood by referring to FIGS. 1-8 of the drawings, like numerals being used for like and corresponding parts of the various drawings. Other features and advantages of the disclosed embodiments will be or will become apparent to one of ordinary skill in the art upon examination of the following figures and detailed description. It is intended that all such additional features and advantages be included within the scope of the disclosed embodiments, and protected by the accompanying drawings. Further, the illustrated figures are only exemplary and not intended to assert or imply any limitation with regard to the environment, architecture, or process in which different embodiments may be implemented. Accordingly, the described aspect is intended to embrace all such alterations, modifications, and variations that fall within the scope and novel idea of the present invention.

Aspects of the present teachings may be further understood in light of the following examples, which should not be construed as limiting the scope of the present teachings in any way.

When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. For example, “a primer” means that more than one primer can, but need not, be present; for example but without limitation, one or more copies of a particular primer species, as well as one or more versions of a particular primer type, for example but not limited to, a multiplicity of different forward primers. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

That is, the terms “including”, “includes”, “having”, “has”, “with”, or variants thereof are used in the detailed description and/or the claims to denote non-exhaustive inclusion in a manner similar to the term “comprising”.

Furthermore, “exemplary” is merely meant to mean an example, rather than the best.

It is also to be appreciated that features, layers and/or elements depicted herein are illustrated with particular dimensions and/or orientations relative to one another for purposes of simplicity and ease of understanding, and that the actual dimensions and/or orientations may differ substantially from that illustrated. That is, in the drawings, the size and relative sizes of layers, regions and/or other elements may be exaggerated or reduced for clarity. Like numbers refer to like elements throughout and explanations that duplicate one another will be omitted. Now, the present invention will be described in detail with reference to the accompanying drawings.

Words such as “thereafter,” “then,” “next,” etc. are not intended to limit the order of the processes; these words are simply used to guide the reader through the description of the methods.

It will be understood that, although the terms first, second, etc. may be used herein to describe various elements, these elements should not be limited by these terms. These terms are only used to distinguish one element from another. For example, a first region/layer could be termed a second region/layer, and, similarly, a second region/layer could be termed a first region/layer without departing from the teachings of the disclosure.

The following terms, as used in the present specification and claims, are intended to have the meaning as defined below, unless indicated otherwise.

As used herein, the term of “nucleic acid” may refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs). The nucleic acid may be DNA, RNA or any naturally occurring or synthetic modification thereof, and combinations thereof. Preferably however the nucleic acid will be DNA, which may be genomic, or, cDNA, and single or double stranded or in any other form.

As used herein, the nucleic acid may be also referred to as the target nucleic acid (or the target). Target nucleic acids include but are not limited to DNA such as but not limited to genomic DNA, mitochondrial DNA, cDNA and the like, and RNA such as but not limited to mRNA, miRNA, and the like. That is, the term “target nucleic acid” may refer to a polynucleotide sequence that is sought to be amplified and/or quantified. The target polynucleotide can be obtained from any source, and can comprise any number of different compositional components. For example, the target can be nucleic acid (e.g. DNA or RNA), transfer RNA, sRNA, and can comprise nucleic acid analogs or other nucleic acid mimic, though typically the target will be messenger RNA (mRNA) and/or micro RNA (miRNA). The target nucleic acids may be prepared using any manner known in the art.

As used herein, the term “hybridization” may refer to the complementary base-pairing interaction of one nucleic acid with another nucleic acid that results in the formation of a duplex, triplex, or other higher-ordered structure, and is used herein interchangeably with “annealing.”

As used herein, the term “detection” may refer to any of a variety of ways of determining the presence and/or quantity and/or identity of a target polynucleotide.

As used herein, the term “nucleic acid molecule” may refer to a sequence of contiguous nucleotides (riboNTPs, dNTPs or ddNTPs, or combinations thereof) of any length which can encode a full-length polypeptide or a fragment of any length thereof, or which can be non-coding. As used herein, the terms “nucleic acid molecule” and “polynucleotide” can be used interchangeably and include both RNA and DNA

As used herein, the term “target” may refer to a range of molecules including but not limited to a miRNA (or pre-miRNAs), an siRNA, a piRNA, a long non-coding RNA, an mRNA, rRNA, tRNA, hnRNA, cDNA, genomic DNA, and long noncoding RNA (ncRNA).

As used herein, the term “complementary” and “complementarity” are interchangeable and may refer to the ability of polynucleotides to form base pairs with one another. Base pairs are typically formed by hydrogen bonds between nucleotide units in antiparallel polynucleotide strands or regions.

As used herein, the term “real-time” detection in the context of amplification indicates an amplification reaction for which the amount of reaction product, i.e. the amplicon or amplification product, is monitored simultaneously with the reaction progression. During real-time detection, amplification products are monitored and quantitated as the amplification products are generated in the reaction mixture.

As used herein, a “label” refers to a molecular moiety that is detectable or produces a detectable response or signal directly or indirectly, e.g., by catalyzing a reaction that produces a detectable signal. Labels include luminescent moieties (such as fluorescent, bioluminescent, or chemiluminescent compounds), radioisotopes, members of specific binding pairs (e.g., biotin and avidin), enzyme or enzyme substrate, reactive groups, or chromophores, such as a dye or particle that results in detectable color.

As used herein, a “detectable label” refers to a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. The detectable label that may be included in an exemplary embodiment of the present invention may be fluorescent substance or intercalator.

FIG. 1 is a plan view of an apparatus for detecting target nucleic acids in various species according to an exemplary embodiment of the present invention, FIG. 2 is a plan view of an apparatus formed with first and second electrodes for detecting target nucleic acids in various species according to an exemplary embodiment of the present invention, and FIG. 3 is an enlarged perspective view of a target nucleic acid amplifier in an apparatus for detecting target nucleic acids in various species according to an exemplary embodiment of the present invention.

Referring to FIGS. 1, 2 and 3, an apparatus configured to detect multiplex target nucleic acids in real time includes a substrate (100) and two or more target nucleic acid amplifiers (200).

Shapes and materials of the substrate (100) are not particularly restricted, and may include what is known in the art. That is, the substrate (100) may be non-conductive material, and may include any one consisting of a group of glass, ceramic, silicone, Si/SiO2 wafer, polyethylene, polystyrene, polypropylene, polyacrylamide and a combination thereof.

Furthermore, the substrate (100) may preferably take a shape of a plate, may be a bead and may take any shape attached and fixed by an electrode (210, described later) of a target nucleic acid amplifier (200).

The two or more target nucleic acid amplifiers (200) may be formed on the substrate (100) with various types of positions and arrangements. The two or more target nucleic acid amplifiers (200) may include a plurality of target nucleic acid amplifiers. Each of the target nucleic acid amplifiers is formed with an electrode (210), and the electrode (210) is attached with a primer set (220) for amplifying the target nucleic acid.

The apparatus according to the present invention include an electrode (210) immobilized with a set (220) of forward primer and reverse primer for amplifying each of the target nucleic acids.

The electrode (210) may be a material selected from a group consisting of a gold, platinum and indium tin oxide. However, the material is not limited thereto. The electrode (210) is preferably formed at each of the two or more target nucleic acid amplifiers (200), and as shown in FIG. 1, one electrode (210) may be formed at each of the two or more target nucleic acid amplifiers (200), which is adequate for detection of amplified product of target nucleic acid using an optical method.

Furthermore, as illustrated in FIG. 2, the electrode (210) includes a first electrode (211) immobilized with a forward primer and a second electrode (212) immobilized with a reverse primer, where the first and second electrodes (211, 212) may be adjacently arranged in parallel, which is adequate for detection of amplified product of target nucleic acid using an electrical method.

The primer set (220) includes a forward primer and a reverse primer for amplifying each of the target nucleic acids. The primer set (220) is immobilized at each electrode (210) of the two or more target nucleic acid amplifiers (200), and may be immobilized in a predetermined arrangement of one or two diagonal directions or one or two cross directions. Furthermore, a reaction group, selected, for example, from a group consisting of aldehyde, carboxyle, ester, activated ester, amido and a combination thereof, may be attached to each distal end of the forward and reverse primers, and the forward and reverse primers may be immobilized to the electrode (210) via the reactor group.

Alternatively, the reaction group may be coated on a surface of the electrode (210), and a primer free from the reaction group may be immobilized to the surface of the electrode (210), or the reaction group may be attached and immobilized to the surfaces of the primer and the electrode (210).

The number of target nucleic acids included in the two or more target nucleic acid amplifiers (200) may be more than two or less than 1,000, but the number is not limited thereto. The apparatus according to the present invention is such that one substrate (100) is formed thereon with multiplex target nucleic acid amplifiers (200) to allow simultaneously amplify or detect multiplex target nucleic acids using one chip. That is, the two or more target nucleic acid amplifiers (200) can be manufactured in a chip to effectively detect nucleic acids of multiplex species in a small space.

Furthermore, the two or more target nucleic acid amplifiers (200) may be appropriately determined based on the number of types of target nucleic acids desired to be detected. In case there exist several types of target nucleic acids desired to be detected, the target nucleic acid amplifiers (200) as many as desired to be detected may be arranged on the substrate.

To this end, the two or more target nucleic acid amplifiers (200) preferably include the electrode (210) attached with mutually different types of forward and reverse primer sets (220). For example, if four mutually different types of virus DNAs are to be detected, four target nucleic acid amplifiers (200) are arranged on the substrate, and each electrode (210) included in the four target nucleic acid amplifiers (200) is immobilized with forward and reverse primer set (220) capable of amplifying the mutually different types of virus DNAs.

Therefore, according to the present invention, multiplex target nucleic acids can be simultaneously detected in real time on one chip.

At the same time, the target nucleic acid amplifiers (200) may further include dNTP and DNA polymerases. The dNTP and DNA polymerases are intended for amplifying the target nucleic acids by way of PCR method.

Furthermore, the target nucleic acid amplifiers (200) may further include detectable labels for detecting amplification products. The detectable labels may be independently included in the target nucleic acid amplifiers (200) or may be included along with the dNTP and DNA polymerases. The detectable labels included in an exemplary embodiment of the present invention may be fluorescent substance or intercalator.

For example, the detectable label may be selected from a group consisting of exa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, Oregon Green, Oregon Green 488-X, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green, YO-PRO-1, YO-PRO-3, YOYO-1, YOYO-3 thiazole orange or ethidium bromide, but not limited thereto. For example, in case the target nucleic acid amplifier (200) includes ethidium bromide, an amplification product can be easily detected using ultraviolet because the ethidium bromide is inserted into a double stranded nucleic acid amplified by the the target nucleic acid amplifier (200).

The apparatus according to the present invention may further include a voltage supplier controlling a voltage supplied to the electrode of the two or more target nucleic acid amplifiers (200). The control of voltage by the voltage supplier may be individually realized by each of the two or more target nucleic acid amplifiers (200). Target nucleic samples may be differently coupled for each primer set (220) by the voltage control. That is, even if multiplex nucleic acids are supplied, the voltage is individually applied to each of the target nucleic acid amplifiers (200), whereby only a target nucleic sample complementarily coupled to a relevant primer set (220) can be specifically coupled.

To be more specific, the voltage supplier can supply voltages of mutually different capacities or voltages of different intensities to the electrode (210) of the two or more target nucleic acid amplifiers (200). The target nucleic sample supplied to the two or more target nucleic acid amplifiers (200) is charged with a negative charge, such that the target nucleic sample can be specifically coupled to the primer set by controlling the intensity of the applied voltage.

Furthermore, the voltage supplier can sequentially supply a voltage to the electrode (210) of the two or more target nucleic acid amplifiers (200). In case multiplex target nucleic samples are supplied to simultaneously detect amplification products of multiplex target nucleic acids, no competition can be generated among the multiplex target nucleic samples by sequentially and individually controlling the voltage application in the amplification process of target nucleic acids.

Mode for the Invention

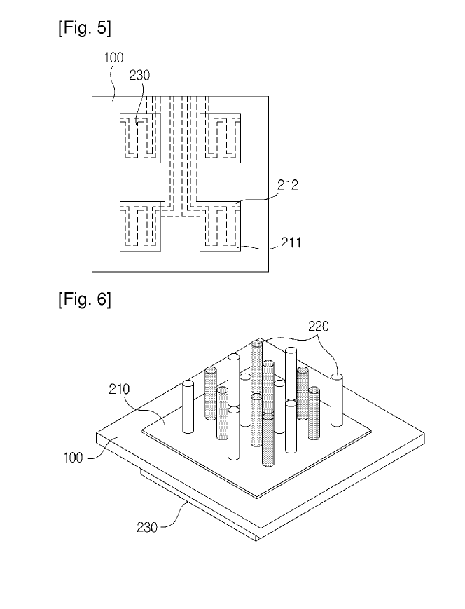

FIG. 4 is a rear view of an apparatus formed with a heat sensor for detecting target nucleic acids in various species according to an exemplary embodiment of the present invention, FIG. 5 is a rear view of an apparatus formed with a heat sensor and first and second electrodes for detecting target nucleic acids in various species according to an exemplary embodiment of the present invention, and FIG. 6 is an enlarged perspective view of an apparatus formed with a heat sensor for detecting target nucleic acids in various species according to an exemplary embodiment of the present invention.

The apparatus thus described for detecting various types of target nucleic acids may further include a heat sensor (230) arranged about the electrode (210) immobilized with the primer set (220).

FIGS. 4 and 5 illustrate a rear surface of the apparatus shown in FIGS. 1 and 2. The heat sensor (230) may be adjacently arranged to the electrode (210) or the electrode (210) may be connected from the adjacent position. Preferably, as shown in FIG. 6, the heat sensor (230) is arranged at a bottom surface of the electrode (210).

The heat sensor (230) may be formed with a material of high heat conductivity capable of sensing and transmitting heat to the electrode. For example, the material may include silver, copper, gold or aluminum but the material is not limited thereto. The heat sensor (230) is arranged about the electrode (210) immobilized with the primer set (220) to control temperatures required for denaturation of target nucleic samples, annealing of primer and polymerization of nucleic acids.

To this end, the apparatus according to the present invention may further include a temperature controller controlling each temperature of the heat sensor (230) at the two or more target nucleic acid amplifiers (200), whereby in case multiplex target nucleic acids are simultaneously detected, and in view of the fact that Tm value of primer immobilized to each target nucleic acid amplifiers (200) may be different, heat sensor (230) is individually controlled to provide an appropriate temperature to an annealing between the target nucleic acid sample and the primer set.

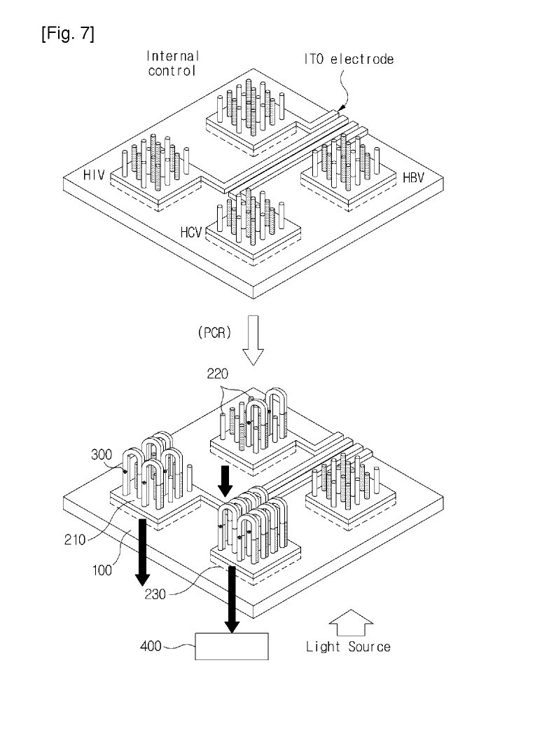

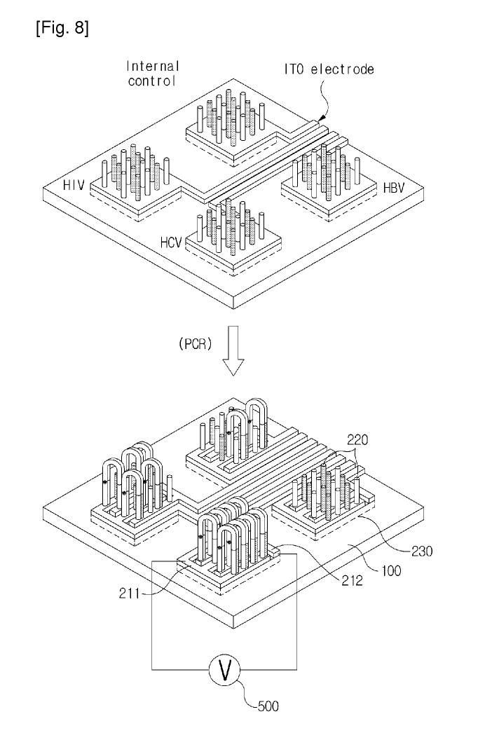

FIGS. 7 and 8 are mimetic diagrams illustrating a method simultaneously detecting target nucleic acids in various species using an apparatus for detecting target nucleic acids in various species according to an exemplary embodiment of the present invention.

That is, FIGS. 7 and 8 illustrate methods simultaneously detecting multiplex target nucleic acids using an apparatus for detecting target nucleic acids in various species according to an exemplary embodiment of the present invention.

Referring to FIGS. 7 and 8, amplification products of target nucleic acids can be detected in real time using an optical method (FIG. 7) or an electrical method (FIG. 8) according to the apparatus according to an exemplary embodiment of the present invention.

Thus, the method for detecting multiplex target nucleic acids in real time may include: contacting various types of target nucleic acid sample to two or more target nucleic acid amplifiers of the claim 1 immobilized with a forward and reverse primer set for amplifying target nucleic acids (S100); applying a voltage to the target nucleic acid amplifiers and controlling the applied voltage to anneal the target nucleic acids and the primer set (S200); amplifying the annealed target nucleic acids (S300); and detecting amplified products of the target nucleic acids in real time (S400).

Now, the method for detecting multiplex target nucleic acids in real time will be described in detail per step.

First of all, the method may include a step of contacting various types of target nucleic acid sample to two or more target nucleic acid amplifiers (200) immobilized with a forward and reverse primer set (220) for amplifying target nucleic acids and a reverse primer (S 100). At this time, the two or more target nucleic acid amplifiers (200) preferably include an electrode attached with mutually different forward and reverse primer sets.

Furthermore, the two or more target nucleic acid amplifiers may further include a heat sensor (230) arranged about the electrode (210) immobilized with the primer set (220). Contact is preferably performed in vitro, where a target nucleic acid desired to be detected is extracted from a cell, a tissue, an organ or an individual which is solved in buffer solution well known in the art and contacted to the target nucleic acid amplifier (200).

Successively, the method may include a step of applying a voltage to the electrode (210) of target nucleic acid amplifiers (200) and controlling the applied voltage to anneal the target nucleic acids (200) and the primer set (220) (S200), where the control of voltage is preferably and individually realized by the two or more target nucleic acid amplifiers (200). For example, the control of voltage may be control of capacity or intensity of the voltage applied to each electrode of the two or more target nucleic acid amplifiers (200) and may be control of sequence of voltage individually applied to each of the electrode (210). Furthermore, the annealing is to individually apply heat to the heat sensor (230) included in each of the two or more target nucleic acid amplifiers (200) and to perform under mutually different temperature conditions.

Thereafter, the method may include the step of amplifying the annealed target nucleic acids and primer set (S300). The amplification process means repetition of denaturation of nucleic acids, annealing of primer and polymerization of nucleic acids, which is well known in the art, so that further discussion thereto will be omitted.

Successively, the method may further include the step of detecting amplification products of the target nucleic acids in real time (S400). The detection may be an optical detection or an electrical detection. A detector used for electrical detection may include a detector for detecting any one or more selected from a group consisting of a current, a voltage, a resistor and impedance, for example. A detector used for optical detection may be a detector for detecting any one or more selected from a group consisting of absorption, transmission, scattering, fluorescence, FRET (Foster Resonance Energy Transfer), surface Plasmon resonance, SERS (Surface Enhanced Raman Scattering), and diffraction. The present invention can simultaneously detect multiplex target nucleic acids in real time using the above mentioned method.

That is, if the target nucleic acid amplifier (200) according to the present invention is manufactured in the optical method as depicted in FIGS. 1 and 4, and if a fluorescent material (300) or intercalator such as SYBR green is used in the detection process as shown in FIG. 7, the amplification product of target nucleic acid amplified by the target nucleic acid amplifier (200) can be optically detected. That is, the fluorescent material (300) or the intercalator may be included in double stranded DNA generated in the amplification process, where the fluorescence generated in the process can be detected by a detector such as a fluorescence measurer (400) in real time.

Furthermore, if the target nucleic acid amplifier (200) is manufactured by the method as shown in FIGS. 2 and 5, and in view of the fact that a first electrode (211) and a second electrode (212) are immobilized with forward and reverse primer set (220) capable of amplifying each nucleic acid, and if the target nucleic acid is amplified to be a double stranded DNA, an amount of current is increased by conductivity of the double stranded DNA, and the amplification product can be detected in real time using a detector such as a current measurer (500).

The previous description of the present invention is provided to enable any person skilled in the art to make or use the invention. Various modifications to the invention will be readily apparent to those skilled in the art, and the generic principles defined herein may be applied to other variations without departing from the spirit or scope of the invention. Thus, the invention is not intended to limit the examples described herein, but is to be accorded the widest scope consistent with the principles and novel features disclosed herein.

Industrial Applicability

The present invention has an industrial applicability in that various types of target nucleic acids can be simultaneously detected in real time to allow manufacturing an apparatus in the shape of a chip, and to efficiently detect a plurality of species of nucleic acids in a small space.

1. An apparatus configured to detect multiplex target nucleic acids in real time, the apparatus characterized by: a substrate; two or more target nucleic acid amplifiers including an electrode attached with a set of a forward primer for amplifying target nucleic acids and a reverse primer.

2. The apparatus of claim 1, characterized in that the electrode includes a first electrode immobilized with the forward primer, and a second electrode immobilized with the reverse primer, wherein the first and second electrodes are adjacently arranged in parallel.

3. The apparatus of claim 1, characterized in that the two or more target nucleic acid amplifiers include an electrode attached with a set of different types of forward primers and reverse primers.

4. The apparatus of claim 1, characterized in that the target nucleic acid amplifiers further include dNTP and DNA polymerase.

5. The apparatus of claim 4, characterized in that the target nucleic acid amplifiers further include detectable labels.

6. The apparatus of claim 1, further characterized by a voltage supplier supplying voltages of mutually different capacities to the electrode of the two or more target nucleic acid amplifiers.

7. The apparatus of claim 1, further characterized by a voltage supplier sequentially supplying a voltage to the electrode of the two or more target nucleic acid amplifiers.

8. The apparatus of claim 1, further characterized by a heat sensor arranged about the electrode immobilized with the primer set.

9. The apparatus of claim 8, characterized in that the two or more target nucleic acid amplifiers further include a heat sensor arranged at a bottom surface of the electrode immobilized with the primer set.

10. The apparatus of claim 9, further characterized by a temperature controller controlling each temperature of the heat sensor at the two or more target nucleic acid amplifiers.

11. A method for detecting multiplex target nucleic acids in real time, the method characterized by: contacting various types of target nucleic acid sample to two or more target nucleic acid amplifiers of the claim 1 immobilized with a forward and reverse primer set for amplifying target nucleic acids; applying a voltage to the target nucleic acid amplifiers and controlling the applied voltage to anneal the target nucleic acids and the primer set; amplifying the annealed target nucleic acids; and detecting amplification products of the target nucleic acids in real time.

12. The method of claim 11, characterized in that the two or more target nucleic acid amplifiers include an electrode attached with mutually different forward and reverse primer sets.

13. The method of claim 11, characterized in that the detection is an optical detection or an electrical detection.

14. The method of claim 11, characterized in that the control of voltage is performed by individual realization at the two or more target nucleic acid amplifiers.

15. The method of claim 14, characterized in that the control of voltage is a control of capacity or intensity of the voltage.

16. The method of claim 14, characterized in that the control of voltage is a control of sequence of the voltage individually applied to the two or more target nucleic acid amplifiers.

17. The method of claim 11, characterized in that the two or more target nucleic acid amplifiers further include a heat sensor arranged about the electrode immobilized with the primer set.

18. The method of claim 17, characterized in that the annealing step includes individually applying heat to the heat sensor included in the two or more target nucleic acid amplifiers to perform the annealing under mutually different temperature conditions.
